(19) United States
(12) Patent Application Publication
(10) Pub. No.: US 2026/0240435 A1
(43) Pub. Date: Aug. 20, 2026

(54) CENTRALIZED ACCESS FOR INTRAORAL SCAN PORTALS

(71) Applicant: IOScans.com LLC, Glendora, CA (US)

(72) Inventor: Saro Hatzakortzian, Glendora, CA (US)

(21) Appl. No.: 19/537,891

(22) Filed: Feb. 12, 2026

Related U.S. Application Data
(60) Provisional application No. 63/758,989, filed on Feb. 14, 2025.

(51) Int. Cl. A61B 5/00; A61B 1/24; A61C 13/00

(52) U.S. Cl. CPC A61B 5/0088; A61B 1/24; A61C 13/0004

(57) ABSTRACT

The disclosed platform comprises a centralized web application that consolidates intraoral scan portals from multiple manufacturers into a single access point for dental laboratories. This system addresses several challenges associated with existing manufacturer-specific portals, including the need for multiple logins, manual download processes, lack of integration, and inconsistencies in data management. By providing a unified platform, the present disclosure enables users to automatically retrieve, organize, and store intraoral scans from various sources, significantly improving workflow efficiency and standardizing data management practices across different scanner manufacturers.

### CROSS REFERENCE TO RELATED APPLICATIONS

This U.S. Non-Provisional Patent Application claims the benefit of U.S. Provisional Patent Application No. 63/758,989, filed on February 14, 2025, titled “CENTRALIZED ACCESS FOR INTRAORAL SCAN PORTALS”, by inventor Saro Hatzakortzian, the contents of which are expressly incorporated herein by this reference as though set forth in their entirety and to which priority is claimed.

### FIELD OF ART

The present disclosure relates to the field of dental technology, specifically to systems and methods for managing intraoral scan data. More particularly, it pertains to centralized web applications designed to consolidate and streamline access to intraoral scan portals from multiple manufacturers, thereby enhancing the efficiency and usability of dental labs.

### BACKGROUND

In the realm of dental technology, intraoral scanning has become a pivotal component, enabling precise and efficient digital impressions for various dental procedures. These scans are typically managed through manufacturer-specific portals, each requiring unique user accounts and interfaces. While these portals serve their intended purpose, they present several challenges that hinder optimal workflow efficiency for dental laboratories.

Prior to the present disclosure, the industry predominantly relied on these manufacturer-specific portals for managing intraoral scans. The existing solutions posed several significant challenges:

Multiple Logins: Dental laboratories were required to create and maintain separate accounts for each manufacturer's scanning portal. This not only increased the administrative burden but also led to inefficiencies in managing multiple credentials.

Manual Download Process: Users had to navigate through different interfaces for each portal and manually download scanned files. This process was time-consuming and prone to errors, further complicating the workflow.

Lack of Integration: There was no unified system available to automatically retrieve, organize, and store scans from multiple sources. This lack of integration meant that dental labs had to manually consolidate data from various portals, leading to fragmented workflows and potential data management issues.

Inconsistencies in Data Management: Different scanner manufacturers employed unique file formats, storage methods, and retrieval processes. This lack of standardization made it challenging to streamline workflows and ensure consistent data management practices across different systems.

Thus, what is needed is a more efficient and integrated solution to manage intraoral scans from multiple manufacturers.

### SUMMARY

To minimize the limitations in the cited references, and to minimize other limitations that will become apparent upon reading and understanding the present specification, the system and method for delivery metric analysis and notification disclosed herein relates to a system and method for centralizing intraoral scans related to dentistry.

One embodiment may be a method for manufacturing dental prosthesis comprising the steps of providing a universal access point, prompting by the universal access point a user to enter one or more log-in credentials into the universal access point, wherein the one or more log-in credentials may comprise a first log-in credential and a second log-in credential, wherein the first log-in credential corresponds to a first intraoral scan portal, and wherein the second log-in credential corresponds to a second intraoral scan portal. The method may comprise the steps of retrieving a first intraoral data from the first intraoral scan portal to the universal access point and retrieving a second intraoral data from the second intraoral scan portal to the universal access point. The method may comprise the step of grouping the first intraoral data and the second intraoral data into a consolidated data. The method may comprise the step of transmitting the consolidated data to a design portal. The method may comprise that the design portal converts the consolidated data into a prosthesis design. The method may comprise that the design portal transmits the prosthesis design to a manufacturing entity. The method may comprise that the manufacturing entity manufactures a prosthesis product based on the prosthesis design. The method may comprise that the prosthesis product is shipped to the user. The method may comprise that the one or more log-in credentials may comprise a third log-in credential and wherein the third log-in credential corresponds to a third intraoral scan portal. The method may comprise the step of retrieving one or more updated intraoral scans from the first intraoral scan portal and the second intraoral scan portals. The method may comprise the step of generating an updated consolidated data based on the one or more updated intraoral scans. The method may comprise the step of transmitting the updated consolidated data to the design portal, wherein the design portal converts the updated consolidated data into an updated prosthesis design. The method may comprise that the design portal transmits the updated prosthesis design to a manufacturing entity. The method may comprise that the manufacturing entity manufactures an updated prosthesis product based on the updated prosthesis design. The method may comprise that the updated prosthesis product is shipped to the user. The method may comprise that accessing the universal access point may comprise entering a universal log-in credential. The method may comprise that the universal access point may comprise a standardized data organization and compatibility module configured to automatically sort and categorize the first intraoral data and the second intraoral data based on patient name, scan date, and case ID. The method may comprise that the universal access point may comprise an enhanced security and compliance system configured to ensure secure encrypted data transfers while maintaining compliance with HIPAA and other industry standards for patient data protection.

The present disclosure is a centralized platform that provides universal access to intraoral scanning portals from multiple manufacturers, requiring users to log in only once to access all scans. It automates the retrieval and download of scans through API integrations, allowing for batch downloading and reducing manual effort. The system organizes and categorizes scans by patient name, scan date, or case ID, ensuring compatibility across different scanning systems and standardizing file formats for lab use. The present disclosure prioritizes security with encrypted data transfers and compliance with HIPAA and other industry standards. Additionally, it optimizes workflow by integrating with third-party dental lab management systems and cloud storage platforms for seamless transfer, automatic backup, and remote accessibility of intraoral scans.

One embodiment may comprise: Centralized Multi-Manufacturer Access, which may act as a universal access point for all intraoral scanning portals, and users may be able to log in once and gain access to scans from multiple manufacturers, reducing redundancy and login fatigue. Another feature may be Automated Scan Retrieval & Download Management which may allow fetching of scans from different manufacturer portals using API integrations and allow for batch downloading, eliminating the need for users to retrieve files manually. Another feature may be Standardized Data Organization & Compatibility, which may include scans from multiple sources that are automatically sorted and categorized based on patient name, scan date, or case ID, and the platform may ensure compatibility across different scanning systems, providing labs with standardized file formats. Another feature may be Enhanced Security & Compliance, which may ensure secure, encrypted data transfers while maintaining compliance with HIPAA and other industry standards for patient data protection. Another feature may be Workflow Optimization & API Integrations, which may integrate with third-party dental lab management systems, allowing seamless transfer of intraoral scans into existing workflows and may connect with cloud storage platforms, enabling automatic backup and remote accessibility.

The present platform may enhance efficiency, usability, security, and cost-effectiveness for dental labs, clinics, and practitioners by consolidating multiple manufacturer intraoral scan portals into a single interface.

By providing a single, automated, and secure hub for managing intraoral scans, the present platform may offer advantages in efficiency, cost-effectiveness, data security, and ease of use.

In one embodiment, the platform may have various components. One component may be a User Interface (Dashboard), which may act as the central hub for users to access all connected intraoral scanner portals; display real-time scan availability, status updates, and a download manager; and provide search and filtering options for locating specific scans. One component may be Multi-Manufacturer API Integration, which may establish secure connections with different intraoral scanner portals by using manufacturer-specific API endpoints to authenticate and retrieve scan data; standardize communication protocols to ensure compatibility with different systems; and automate the fetching, downloading, and categorization of scans. One component may be Authentication & Security Layer. A Single Sign-On (SSO) may allow users to log in once and access multiple scanner portals, provide Data Encryption that may ensure secure transfer of patient scans, complying with HIPAA and industry regulations; and provide Role-Based Access Control (RBAC) to Grant different permission levels for users (e.g., lab technicians, managers). One component may be Scan Data Management & Standardization, which includes Auto-Sorting to organize scans by patient name, case ID, scan date, and manufacturer, File Format Compatibility to convert and normalize scans into universally accepted formats for various lab workflows, and Storage Integration to allow users to store scans on local servers or cloud platforms like Google Drive, Dropbox, and AWS. One component may be Automated Notifications & Alerts, which notifies users when new scans are available for download, sends customized alerts for urgent cases or missing scans, and integrates with email or SMS for real-time updates. Integration with lab management software may allow users to automatically send scans, track orders by syncing with case tracking systems, and automate workflows to reduce manual data entry, saving time and minimizing errors.

In one embodiment, the various components may interact as follows: A dental lab technician logs into the platform using SSO, and the system verifies credentials to grant access to connected scanner portals. The platform automatically and securely retrieves available scans from all linked manufacturer portals, ensuring privacy compliance. The system auto-sorts scans by patient and case details, converting them into compatible formats for seamless lab workflow integration. The dashboard provides real-time updates, allowing users to search, filter, batch-download scans, and export directly to lab software or cloud storage. All scan data is encrypted during transfer and storage, with access logs tracking user activity for security auditing. Users receive alerts for new scans, and the system integrates with dental lab management software to streamline case processing.

In some embodiments, ways to notify users of new scans may include the current preferred method of email and SMS alerts. Alternative methods could involve push notifications via a mobile app, allowing users to receive instant notifications on their mobile devices when new scans are available, with the advantage of real-time updates but requiring mobile app development. Another alternative could be automated voice call notifications, where a voice AI system calls lab technicians to notify them of scan availability, which is useful for urgent cases but may be unnecessary for most users and disruptive in busy work environments.

Other features and advantages inherent in the disclosed system and method for delivery metric analysis and notification, besides those which are claimed and disclosed, will become apparent to those skilled in the art from the following detailed description and its accompanying drawings.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps which are illustrated. When the same numeral appears in different drawings, it refers to the same or like components or steps.

FIG. 1 is a flow diagram of one embodiment of a method for manufacturing dental prosthesis.

FIG. 2 is a block diagram of one embodiment of a system for gathering and using intraoral scans.

FIG. 3 is a workflow showing one embodiment of the present disclosure.

FIG. 4 is a system workflow of one embodiment of the present disclosure.

### DETAILED DESCRIPTIONOF THE ILLUSTRATIVE EMBODIMENTS

In the following detailed description, numerous specific details are set forth in order to provide a thorough understanding of various aspects of one or more embodiments. However, these embodiments may be practiced without some or all of these specific details. In other instances, well-known methods, procedures, and/or components have not been described in detail so as not to unnecessarily obscure aspects of embodiments.

The present platform introduces a centralized web application that consolidates intraoral scan portals from multiple manufacturers into a single access point for dental labs. This streamlined workflow eliminates the need for users to log in separately to various manufacturer-specific portals, enhancing efficiency and usability.

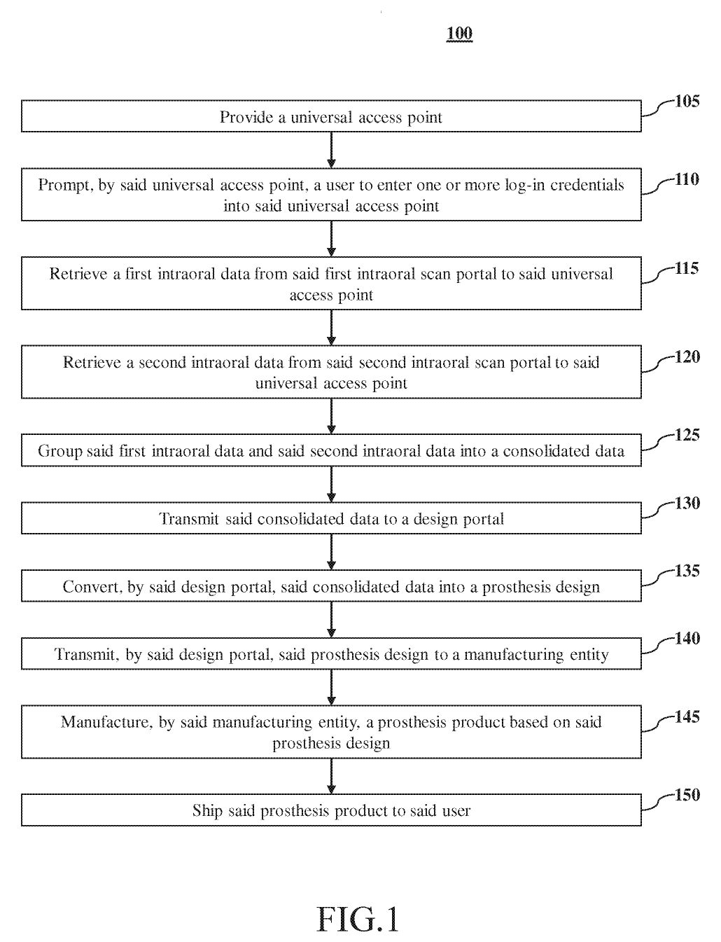

FIG. 1 is a flow diagram of one embodiment of a method for manufacturing dental prosthesis. As shown in FIG. 1, the method for accessing intraoral scan portals 100 may comprise the steps: providing a universal access point 105; prompting, by the universal access point, a user to enter one or more log-in credentials into the universal access point 110; retrieving a first intraoral data from the first intraoral scan portal to the universal access point 115; retrieving a second intraoral data from the second intraoral scan portal to the universal access point 120; grouping the first intraoral data and the second intraoral data into a consolidated data 125; transmitting the consolidated data to a design portal 130; converting, by the design portal, the consolidated data into a prosthesis design 135; transmitting, by the design portal, the prosthesis design to a manufacturing entity 140; manufacturing, by the manufacturing entity, a prosthesis product based on the prosthesis design 145; and shipping the prosthesis product to the user 150.

In some embodiments, the one or more log-in credentials comprise a first log-in credential, and a second log-in credential. In other embodiments, the log-in credentials comprise a third log-in credential. In yet other embodiments, the log-in credentials may comprise a greater number of log-in credentials, the number of which is not limited by the present disclosure, but by technical limitations of third-party applications.

In a preferred embodiment, the log-in credentials correspond to intraoral scan portals, such that each set of log-in credentials allows access to a different intraoral scan portal, or set of intraoral scan portals. In some embodiments, the intraoral scan portal may be substantially any electronically stored data relating to oral scanning. Oral scans may encompass various types of imaging techniques used to capture detailed images of the mouth, teeth, and surrounding structures. These include intraoral scanners, which create digital impressions of the teeth and gums; panoramic X-rays, providing a broad view of the entire mouth; cone beam computed tomography (CBCT), offering 3D images for precise diagnostics; and traditional dental X-rays, which capture specific areas for detailed examination. In some embodiments, each of the intraoral scan portals comprises data generated from different oral scans, or may include scans from different times that may be used to determine trajectory of oral health of the individual.

In a preferred embodiment, accessing the universal access point comprises entering a universal log-in credential, such that the user may enter their universal log-in credential into the universal access point and thereby be able to access all, or some, of the intraoral scan portals to which the user’s log-in credentials relate. In this way, each time the user accesses the universal access point, the user may thereby have access to the data generated from different scans. In the event that one or more scans on the intraoral scan portals have been updated, revised, or there are new scans, those updated, revised, or new scans may be retrieved by the universal access point and used to generate updated prosthesis designs, which may then be used to manufacture new prosthesis to be shipped to the user, or their patient.

In some embodiments, each of the intraoral scan portals may correspond to a different manufacturer or service provider.

In some embodiments, the user may be a dental or oral practitioner, and may use the universal access point to track data from one or more patients. Scan related to each patient may be stored separately and accessed separately, such that the practitioner may be able to quickly see updates to their patients’ status in the event that the patient has scans performed by third parties.

In one embodiment, all scan data relating to a specific patient may be retrieved from different intraoral scan portals and consolidated on the universal access point or application.

In some embodiments, the universal access point may be considered an application and/or a platform. In a preferred embodiment, the user interacts with the universal access point in order to obtain and/or process intraoral scan data from various intraoral scan portals. In some embodiments, the user may be able to add one or more intraoral scan data from a local source into the universal access point.

In one embodiment, the universal access point automatically periodically requests updated information from the intraoral scan portals. In some embodiments, this process may be triggered by a user to ensure that up to date scans are used for various processes.

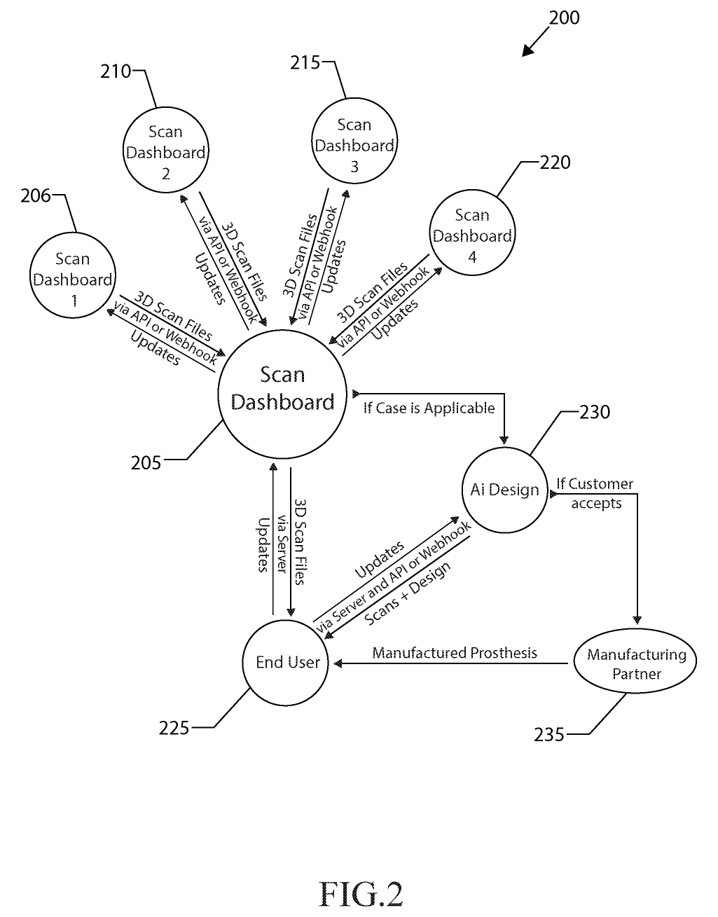

FIG. 2 is a block diagram of one embodiment of a system for gathering and using intraoral scans. As shown in FIG. 2, one embodiment of a system for gather and using intraoral scans 200 may comprise universal access point 205; first intraoral scan portal 206; second intraoral scan portal 210; third intraoral scan portal 215; fourth intraoral scan portal 220; user 225; design engine 230; and manufacturer 235.

User 225 may access universal access point 205 by providing universal log-in credentials. User 225 may then input one or more log-in credentials into universal access point 205 that correspond to one or more intraoral scan portals 206, 210, 215, 220. After the log-in credentials are input, validated, and/or approved, intraoral scan portals 206, 210, 215, 220 may send and/or receive information and/or data, and the information and/or data may be stored within universal access point 205. The information and/or data may comprise intraoral scans previously stored or uploaded to various intraoral scan portals 206, 210, 215, 220.

User 225 may then access this information and/or data through universal access point 205. User 225 may instruct universal access point 205 to transfer the information and/or data to a design engine 230. Design engine 230 may be configured to convert the information/data to a dental or oral prosthesis design. This prosthesis design may be approved by user 225 and/or universal access point 205, and sent to manufacturer 235. Manufacturer 235 to manufacture the prosthesis, and ship the prosthesis to the user. Manufacturer 235 may be substantially any entity that is capable of manufacturing dental prostheses.

In some embodiments, intraoral scan portals 206, 210, 215, 220 may send updated information/data to universal access point 205 upon request or of their own initiative. In some embodiments, any updated information may trigger one or more notifications to each affected entity or system to evaluate whether new information should or needs to be integrated into the process of generating the prosthesis.

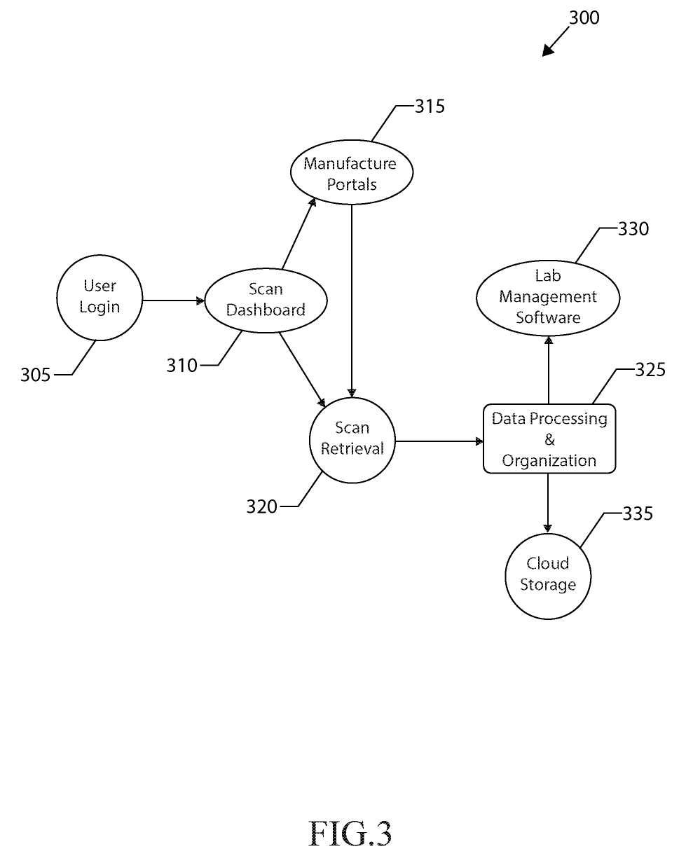

FIG. 3 is a workflow showing one embodiment of the present disclosure. As shown in FIG. 3, the workflow 300 may comprise a user log-in 305 into a Scan Dashboard 310, which may interface directly with manufacturer portals 315 and conduct scan retrieval 320 from data processing & organization 325 entities, such as lab management software 330 and cloud storage 335, which may each be maintained by different providers.

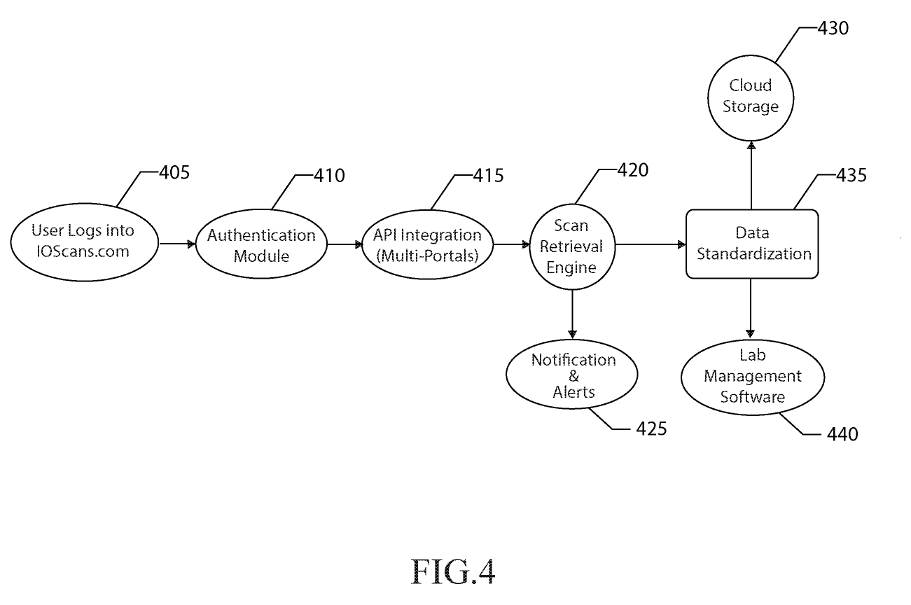

FIG. 4 is a system workflow of one embodiment of the present disclosure. The workflow 400 may comprise user logging into a scan dashboard 405. The scan dashboard 405 may include API integration into multiple portals 415 to allow a scan retrieval engine 420 to function. The scan retrieval engine 420 may receive and/or send notifications and alerts 425. The scan retrieval engine 520 may preferably receive data in a standard format 435, or convert non-standard data into standardized data format, which may retrieve and/or send data to cloud storage 430 and/or lab management software 440.

One embodiment of the present disclosure may comprise a User Interface (UI) & Dashboard, which is a web-based platform where users log in and manage intraoral scans, displaying available scans, retrieval progress, and notifications; an Authentication & Security Module that uses Single Sign-On (SSO) or OAuth authentication to allow users to access multiple manufacturer portals with a single login and ensures HIPAA compliance for patient data security; an API Integration Module that establishes secure API connections with intraoral scanner manufacturer portals, fetches new scans automatically, and standardizes file formats; a Scan Retrieval & Processing Engine that downloads available scans from multiple sources and categorizes scans based on patient name, case ID, scan date, and scanner type; a Data Standardization & Storage System that converts different scanner file formats into a universally accepted format and allows users to store scans locally or in cloud storage; a Lab Management System Integration that exports scans directly into lab management software for case tracking; and Automated Notifications & Alerts that send email/SMS alerts when new scans are available and track pending cases, reminding users of incomplete downloads.

The User Interface (Dashboard) may provide a centralized platform for managing scans. Authentication & Security may ensure secure and streamlined access to multiple portals. The API Integration Module may automate scan retrieval from different manufacturer systems. Scan Retrieval & Processing may download, organize, and standardize scans. Data Standardization & Storage may convert files into compatible formats and organize them. Lab Management Integration may streamline workflow by sending scans directly to lab software. Automated Notifications may notify users about new scans and pending cases.

Some of the components of the present disclosure may interact with one another as follows: User logs in to the present platform via a web-based dashboard. The Authentication Module connects the user to all linked scanner portals. The API Integration Module retrieves available scans from each manufacturer. The Scan Retrieval Engine downloads and organizes the scans. The Data Standardization System ensures all scans are in a consistent format. Scans are stored and sent to lab management software or cloud storage. The Notification System alerts users to new scans or pending cases.

FIG. 5 is a workflow diagram summarizing how the present platform interacts with different scanner portals and lab software.

In one embodiment, a Preferred Workflow Diagram for the present platform illustrating how the system components interact may comprise the following sequence. The user logs into the present platform through secure single sign-on authentication. The Authentication Module verifies user access and links to manufacturer portals. The API Integration Module fetches scans from different scanner providers. The Scan Retrieval Engine downloads and organizes available scans. Data Standardization converts different scan formats into a unified system. Cloud Storage & Lab Management Integration sends scans to cloud storage or directly to lab software. Finally, the Notification System alerts users when new scans are available.

One embodiment of the present disclosure may comprise the following steps. The user logs in to the present platform using a single set of credentials. The platform connects securely to all linked scanner portals. The system automatically fetches all newly available scans. The scans are categorized by patient name, case ID, and date. Users can download scans, store them in the cloud, or send them directly to lab management software. Users receive email/SMS notifications when scans are ready.

One embodiment of the present disclosure may comprise a web-based platform using API integrations to retrieve scans from multiple manufacturers, where scans are automatically sorted, standardized, and stored, allowing users to log in once to manage all connected scanner portals. Alternative approaches include desktop-based software instead of a web platform, which could be installed locally on lab computers, offering increased data security and offline access but limited accessibility and requiring installation and maintenance. Another approach is manual upload instead of API retrieval, which would allow users to manually upload scans to the platform, making it easier to implement with manufacturers that lack APIs but requiring user intervention and reducing efficiency. Additionally, the platform could operate as a middleware plugin for existing lab management software, eliminating the need for a separate interface but requiring compatibility with multiple lab software solutions.

In some embodiments, possible enhancements may include AI-based scan categorization, where machine learning analyzes scan data and automatically assigns case types. Blockchain-based data security could be implemented by storing scan access logs on a blockchain ledger, enhancing traceability and security. Additionally, a mobile app version could be developed, allowing users to review scans on mobile devices, thereby increasing flexibility for on-the-go use.

While multiple embodiments are disclosed, still other embodiments will become apparent to those skilled in the art from the following detailed description. As will be realized, these embodiments are capable of modifications in various obvious aspects, all without departing from the spirit and scope of protection. Accordingly, figures and the detailed descriptions thereof are to be regarded as illustrative in nature and not restrictive. Also, the reference or non-reference to a particular embodiment shall not be interpreted to limit the scope of protection.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 5% of the indicated number or range of numbers. In one embodiment, the term “approximately” and “about,” may refer to a deviance of between 0.0001-10% from the indicated number or range of numbers.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

As will be appreciated by one skilled in the art, the systems and methods may take the form of an entirely hardware embodiment, an entirely software embodiment, or an embodiment combining software and hardware embodiments. Furthermore, the systems and methods may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, compact discs, read-only-memory (CD-ROMs), optical storage devices, or magnetic storage devices.

Embodiments of the systems and methods are described below with reference to schematic diagrams, block diagrams, and flowchart illustrations of methods, systems, apparatuses, and computer program products. It will be understood that each block of the block diagrams, schematic diagrams, and flowchart illustrations, and combinations of blocks in the block diagrams, schematic diagrams, and flowchart illustrations, respectively, may be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that may direct a computer or other programmable data processing apparatus to function in a particular manner. In some embodiments, computer program instructions may be cloud based or edge based, wherein data related thereto may also be stored in the cloud. In some embodiments, computer program instructions and related data may be stored differently, such that one is stored locally or on a controlled server, whereas the other may be stored in the cloud or edge-based systems. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Unless otherwise stated, all measurements, values, ratings, positions, magnitudes, sizes, locations, and other specifications, which set forth in this specification, including in the claims that follow, are approximate, not exact. They are intended to have a reasonable range, which is consistent with the functions to which they relate and with what is customary in the art to which they pertain.

In addition, the various illustrative logical blocks, modules, and circuits described in connection with certain embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, system-on-a-chip, or state machine. A processor may also be implemented as a combination of computing devices, e.g., a combination of a DSP and a microprocessor, a plurality of microprocessors, one or more microprocessors in conjunction with a DSP core, or any other such configuration.

Operational embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, a DVD disk, or any other form of storage medium known in the art. An exemplary storage medium is coupled to the processor such the processor may read information from, and write information to, the storage medium. In the alternative, the storage medium may be integral to the processor. The processor and the storage medium may reside in an ASIC or may reside as discrete components in another device.

Furthermore, the one or more versions may be implemented as a method, apparatus, or article of manufacture using standard programming and/or engineering techniques to produce software, firmware, hardware, or any combination thereof to control a computer to implement the disclosed embodiments. Non-transitory computer readable media may include but are not limited to magnetic storage devices (e.g., hard disk, floppy disk, magnetic strips), optical disks (e.g., compact disk (CD), digital versatile disk (DVD)), smart cards, and flash memory devices (e.g., card, stick). Those skilled in the art will recognize many modifications may be made to this configuration without departing from the scope of the disclosed embodiments.

The foregoing description of the preferred embodiment has been presented for the purposes of illustration and description. While multiple embodiments are disclosed, still other embodiments will become apparent to those skilled in the art from the above detailed description, which shows and describes the illustrative embodiments. As will be realized, these embodiments are capable of modifications in various obvious aspects, all without departing from the spirit and scope of the present disclosure. Accordingly, the detailed description is to be regarded as illustrative in nature and not restrictive. Also, although not explicitly recited, one or more additional embodiments may be practiced in combination or conjunction with one another. Furthermore, the reference or non-reference to a particular embodiment shall not be interpreted to limit the scope of protection. It is intended that the scope of protection not be limited by this detailed description, but by the claims and the equivalents to the claims that are appended hereto.

Except as stated immediately above, nothing which has been stated or illustrated is intended or should be interpreted to cause a dedication of any component, step, feature, object, benefit, advantage, or equivalent to the public, regardless of whether it is or is not recited in the claims.

What is claimed is:

1. A method for manufacturing a dental prosthesis, comprising:
providing a universal access point;
prompting, by said universal access point, a user to enter one or more log-in credentials into said universal access point;
wherein said one or more log-in credentials comprise a first log-in credential and a second log-in credential;
wherein said first log-in credential corresponds to a first intraoral scan portal; and
wherein said second log-in credential corresponds to a second intraoral scan portal.

2. The method for manufacturing a dental prosthesis of claim 1, further comprising:
retrieving a first intraoral data from said first intraoral scan portal and accessing said first intraoral data on to said universal access point; and
retrieving a second intraoral data from said second intraoral scan portal and accessing said second intraoral data on to said universal access point.

3. The method for manufacturing a dental prosthesis of claim 2, further comprising:
grouping, by said universal access point said first intraoral data and said second intraoral data into a consolidated data.

4. The method for manufacturing a dental prosthesis of claim 3, further comprising:
transmitting by said universal access point said consolidated data to a design portal.

5. The method for manufacturing a dental prosthesis of claim 4, further comprising converting, by said design portal, said consolidated data into a prosthesis design.

6. The method for manufacturing a dental prosthesis of claim 5, further comprising transmitting, by said design portal, said prosthesis design to a manufacturer.

7. The method for manufacturing a dental prosthesis of claim 6, further comprising, manufacturing, by said manufacturer, a prosthesis product based on said prosthesis design.

8. The method for manufacturing a dental prosthesis of claim 7, further comprising shipping said prosthesis product to said user.

9. The method for manufacturing a dental prosthesis of claim 8, wherein said one or more log-in credentials comprises a third log-in credential; and
wherein said third log-in credential corresponds to a third intraoral scan portal.

10. The method for manufacturing a dental prosthesis of claim 8, further comprising:
retrieving one or more updated intraoral scans from said first intraoral scan portal and said second intraoral scan portals.

11. The method for manufacturing a dental prosthesis of claim 10, further comprising:
generating an updated consolidated data based on said one or more updated intraoral scans.

12. The method for manufacturing a dental prosthesis of claim 11, further comprising:
transmitting said updated consolidated data to said design portal; and
converting, by said design portal, said updated consolidated data into an updated prosthesis design.

13. The method for manufacturing a dental prosthesis of claim 12, further comprising: transmitting, by said design portal, said updated prosthesis design to said manufacturer.

14. The method for manufacturing a dental prosthesis of claim 13, further comprising: manufacturing, by said manufacturer, an updated prosthesis product based on said updated prosthesis design.

15. The method for manufacturing a dental prosthesis of claim 14,further comprising: shipping said updated prosthesis to said user.

16. The method for manufacturing a dental prosthesis of claim 15, further comprising: entering a universal log-in credential in order to open said universal access point.

17. The method for manufacturing a dental prosthesis of claim 1, wherein said universal access point comprises a standardized data organization and compatibility module that is configured to automatically sort and categorize said first intraoral data and said second intraoral data based on patient name, scan date, and case ID.

18. The method for manufacturing a dental prosthesis of claim 1, wherein said universal access point comprises an enhanced security and compliance system configured to ensure secure and encrypted data transfers while maintaining compliance with HIPAA and other industry standards for patient data protection.
